(19) United States
(12) Patent Application Publication
(10) Pub. No.: US 2005/0214894 A1
(43) Pub. Date: Sep. 29, 2005

(54) High throughput assay

(76) Inventors: Christopher J. Schofield, Oxford (GB); Kirsty S. Hewitson, Oxford (GB); Luke A. McNeill, Oxford (GB)

Correspondence Address: NIXON & VANDERHYE, PC, 901 NORTH GLEBE ROAD, 11TH FLOOR, ARLINGTON, VA, 22203, US

(21) Appl. No.: 11/090,320

(22) Filed: Mar. 28, 2005

Related U.S. Application Data
(60) Provisional application No. 60/592,135, filed on Jul. 30, 2004.

(30) Foreign Application Priority Data
Mar. 26, 2004 (GB) 0406914.2
Aug. 26, 2004 (GB) 0419128.4
Nov. 23, 2004 (GB) 0425760.6

(51) Int. Cl. C12Q 1/26

(52) U.S. Cl. 435/25

(57) ABSTRACT

A method for detecting 2-oxoglutarate oxygenase activity, which method comprises: (i) contacting a 2-oxoglutarate oxygenase and a substrate of the 2-oxoglutarate oxygenase in the presence of 2-oxoglutarate; (ii) adding a derivatisation reagent capable of forming a fluorescent product with 2-oxoglutarate; (iii) detecting the fluorescent product produced by the reaction between the derivatisation reagent and 2-oxoglutarate, if any, thereby detecting 2-oxoglutarate oxgenase activity.

### FIELD OF THE INVENTION

The present invention relates to a high throughput assay for detecting 2-oxoglutarate oxygenase activity and to the use of the assay to detect agents which modulate 2-oxoglutarate oxygenase activity. Agents which modulate 2-oxoglutarate oxygenase activity are also provided.

### BACKGROUND TO THE INVENTION

The super-family of 2-oxoglutarate (2-OG) and ferrous iron dependent enzymes catalyse a wide range of oxidative reactions including the hydroxylation of inactivated C—H bonds (such as in the conversion of proline to 3-hydroxyproline as catalysed by proline-3-hydroxylase), desaturation of C—C bonds and oxidative cyclisations.

In almost all cases, enzymes belonging to the super-family of 2-OG oxygenases use 2-OG as a cosubstrate. In these cases the 4-electron oxidising power of a dioxygen molecule is coupled to the two-electron oxidation of the substrate (e.g. proline to 3-hydroxyproline in reactions catalysed by proline-3-hydroxylase) and the oxidation of 2-OG to give succinate and CO₂.

The stoichiometry of a typical hydroxylation reaction as catalysed by a 2-OG oxygenase is thus as follows:

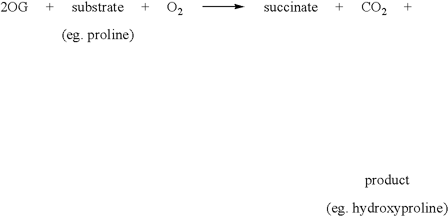

Assays measuring 2-OG consumption are useful for mechanistic studies and in the discovery of compounds that inhibit catalytic activity.

In the most part current assays for 2-OG oxygenases rely on following the consumption of 2-OG by monitoring the release of ¹⁴CO₂ produced from [C-14]-labelled 2-OG. Although widely used by various laboratories, this assay suffers from the drawbacks in that it involves the use of radioactive material with associated cost, handling, safety and health problems. In addition, it is inconvenient and requires the use of specialist equipment. Despite this it is the assay of choice for many laboratories working in the field.

Other assays have been developed that follow the appearance of oxidised product. While some of these follow an easily observed absorbance change, many rely on high performance liquid chromatography, either with or without a derivatisation step, and/or mass spectrometric techniques. Both of these approaches have the disadvantage of being intensive in terms of time, labour, and materials. Further, assays that reply upon monitoring loss of the ‘prime’ substrate or detection of (hydroxylated or oxidised) products are generally only applicable to a specific enzyme.

The number of groups working with inadequate assays, such as those employing detection of radioactive carbon dioxide demonstrates an unmet need for new assays amenable to high-throughput work that are both safer and more efficient. Such high-throughput assays are required to screen collections or libraries of potential inhibitors.

### SUMMARY OF THE INVENTION

The present inventors have developed an alternative assay for 2-OG oxygenase activity that is based on derivatisation of 2-OG. The assay can be used to find inhibitors or activators of the 2-OG oxygenases and can be applied in a high-throughput format using conventional equipment.

More particularly, the inventors have used the new assay to monitor activity of the 2-OG oxygenases known as factor inhibiting hypoxia-inducible factor (FIH) and prolyl hydroxylase domain containing 2 (PHD2) enzyme, and to detect inhibitors of these enzymes. The reaction of FIH with a glutathione-S-transferase tagged fragment of the HIF transactivation domains (residues 786-826) has been previously described using assays that employed detection of radioactive carbon dioxide. The present inventors have shown that the new assay procedure gives the same results as have been achieved previously and that the new assay provides a safer and more efficient alternative to the known assays.

The new assay measures the consumption of 2-OG by a 2-oxoglutarate oxygenase and thus measures catalytic activity of the oxygenase. To do this, the inventors have developed a derivatisation process whereby the 2-OG forms a fluorescent product with ortho-phenylenediamine (OPD), or other suitable derivatisation reagent, whereas other products such as succinate do not. Accordingly, the present invention provides:
a method for detecting 2-OG oxygenase activity, which method comprises the steps of:
(i) contacting a 2-OG oxygenase and a substrate of the 2-OG oxygenase in the presence of 2-OG;
(ii) adding a derivisation reagent capable of forming a fluorescent product with 2-OG;
(iii) detecting the fluorescent product produced by the reaction between the derivatisation reagent and 2-OG, if any, thereby detecting 2-OG oxygenase activity.
a method for determining whether a test agent modulates activity of a 2-OG dependent oxygenase comprising the steps of:
(i) contacting a 2-OG oxygenase, a test agent and optionally a substrate of the 2-OG oxygenase in the presence of 2-OG;
(ii) adding a derivatisation reagent capable of forming a fluorescent product with 2-OG;
(iii) detecting the fluorescent product produced by the reaction between the derivatisation reagent and 2-OG, if any; and
(iv) comparing the amount of fluorescent product detected to the amount of fluorescent product detected in the absence of the test agent,
thereby determining whether the test agent modulates activity of the 2-OG oxygenase;
a method for determining whether a test agent is a selective modulator of a first 2-OG oxygenase, comprising the steps of:
(i) contacting a first 2-OG oxygenase, a test agent and optionally a substrate of the first 2-OG oxygenase in the presence of 2-OG;
(ii) adding a derivatisation reagent capable of forming a fluorescent product with 2-OG;
(iii) detecting the fluorescent product produced by the reaction between the derivatisation reagent and 2-OG, if any; and
(iv) comparing the amount of fluorescent product detected to the amount of fluorescent product detected in the absence of the test agent;
(v) contacting a second 2-OG oxygenase, a test agent and optionally a substrate of the second 2-OG oxygenase in the presence of 2-OG;
(vi) adding a derivatisation reagent capable of forming a fluorescent product with 2-OG;
(vii) detecting the fluorescent product produced by the reaction between the derivatisation reagent and 2-OG, if any; and
(viii) comparing the amount of fluorescent product detected to the amount of fluorescent product detected in the absence of the test agent;
thereby determining whether the test agent is a selective modulator of the first 2-OG oxygenase, wherein the test agent is a selective modulator of the first 2-OG oxygenase if it modulates activity of the first 2-OG oxygenase but does not modulate activity of the second 2-oxoglutarate oxygenase;
an agent which modulates 2-OG oxygenase activity identified by a method according to the invention; and
a method of treating a condition associated with increased or reduced 2-OG oxygenase activity or a condition in which it is desired to modulate 2-OG oxygenase activity, comprising administering a therapeutically effective amount of an agent according to the invention to an individual having the condition.

### BRIEF DESCRIPTION OF THE FIGURES

FIG. 1 shows one reaction of OPD with the α-ketoacid motif of 2-OG to give 3-(2-Carboxyethyl)-2(1H)-quinoxalinone.

FIG. 2 shows the fluorescence spectra of the product of the reaction between OPD and 2-OG (a) Excitation (emission=420nm) and (b) emission (excitation=340nm) spectra of: (1) 125 μM Quinoxalinone in 0.25 M NaOH solution, (2) 250 μM in 0.5 M HCl solution (3) 10 mg/ml OPD in 0.5 M HCl.

FIG. 3 is a graph showing the reaction of OPD with varying concentrations of 2-OG. Concentrations quoted are concentration in 100 μl volume, made as described in the Examples.

FIG. 4 is a chart showing the effect of the assay components on the fluorescence generated during an incubation. Black bars=no incubation, grey bars=15 minute incubation. Buffer=50 mM Tris/HCl pH 7.5.

FIG. 5 shows the dependence of 2-OG consumption on FIH. Initial substrate and 2-OG concentrations were held constant while initial FIH and iron concentrations were varied to keep a constant ratio.

FIG. 6 is a graph showing the dependence of FIH activity on the initial concentration of 2-OG. FIH, iron and prime substrate concentrations were constant, and the incubation time was 15 minutes.

FIG. 7 illustrates the effect of inhibitors (1 mM) on the activity of FIH.

FIG. 8 depicts the effect of various N-oxalyl amino acid inhibitors on the activity of FIH.

FIG. 9 depicts the effect of various N-oxalyl amino acid inhibitors on the activity of PHD2 in both Tris and HEPES buffers.

### BRIEF DESCRIPTION OF THE SEQUENCES

SEQ ID NO: 1 is the amino acid sequence of FIH.

SEQ ID NO: 2 is the amino acid sequence of PHD2.

SEQ ID NO: 3 is the amino acid sequence of a truncated form of PHD2 which retains 2-OG dependent oxygenase activity.

### DETAILED DESCRIPTION OF THE INVENTION

The present inventors have devised a novel high-throughput assay for detecting 2-oxoglutarate (2-OG) dependent oxygenase activity. The terms 2-OG oxygenase and 2-OG dependent oxygenase are used interchangeably herein. The assay typically comprises:
(i) contacting a 2-OG oxygenase and a substrate of the 2-OG oxygenase in the presence of 2-OG;
(ii) adding a derivatisation reagent capable of forming a fluorescent product with 2-OG;
(iii) detecting the fluorescent product produced by the reaction between the derivatisation reagent and 2-OG, if any, thereby detecting 2-OG oxgenase activity.

The assay may further comprise stopping 2-OG oxygenase activity prior to step (ii), for example by adding hydrochloric acid. Step (i) may be carried out in the presence of ferrous iron.

### 2-oxoglutarate Dependent oxygenase

The 2-oxoglutarate (2-OG) oxygenase used in an assay of the invention may be any member of the super-family of 2-OG and ferrous iron dependent enzymes which use 2-OG as a cosubstrate.

2-OG dependent oxygenases that may be used in an assay of the invention include, for example, factor inhibiting hypoxic inducing factor (FIH), prolyl hydroxylase domain (PHD) containing enzymes, collagen prolyl-4-hydroxylase, collagen prolyl-3-hydroxylase, lysyl hydroxylase, aspartyl hydroxylase, phytanoyl coenzyme A hydroxylase, gamma-butyrobetaine hydroxylase, trimethyllysine hydroxylase, AlkB and related 2-OG oxygenases involved in DNA modification, deacetyl cephalosporin C synthase, deacetoxy cephalosporin C synthase, carbapenem synthase and proline-3-hydroxylase.

2-OG dependent oxygenases may be mammalian, preferably human polypeptides. Human 2-OG oxygenases include AlkB, collagen prolyl hydroxylases, lysine hydroxylases, the aspartyl/asparagine hydroxylase known to hydroxylate endothelial growth factor domains, phytanoyl CoA hydroxylase, gamma-butyrobetaine hydroxylase, trimethyl lysine hydroxylase, HIF prolyl hydroxylase isoforms including PHD1, PHD2, PHD3, the phosphatidyserine receptor, Mina53 and enzymes closely related to FIH including those proteins in the SWALL database that are referenced by the following numbers: Q9NWJ5, Q8TB10, Q9Y4E2, O95712, Q9H8B1, Q9NWT6 in Homo sapiens, and Q91W88 and Q9ER15 in Mus musculus and homologues of these enzymes.

The amino acid sequence of FIH is shown in SEQ ID NO:1. The FIH used in a method of the invention may comprise or consist of the sequence shown in SEQ ID NO: 1 or may be a fragment or variant thereof having 2-OG-dependent oxygenase activity.

The fragment of FIH is one which retains 2-OG-dependent oxygenase activity. The fragment is typically from about 150 to 348 amino acids in length, for example, the fragment may be at least about 200, 250, 300, 330, 340 or 348 amino acids in length.

The amino acid sequence of PHD2 is shown in SEQ ID NO: 2. The PHD2 used in a method of the invention may comprise or consist of the sequence shown in SEQ ID NO: 2 or may be a fragment or variant thereof having 2-OG-dependent oxygenase activity.

The fragment of PHD2 is one that retains 2-OG-dependent oxygenase activity. The fragment typically has an improved solubility compared to full-length PHD2. Solubility may be determined using an E.coli expression system. The fragment is typically from about 150 to 425 amino acids in length. For example the fragment may comprise at least about 200, 250, 300, 350, 400 or 420 amino acids of the sequence shown in SEQ ID NO: 2. Preferred fragments are C-terminal fragments. A fragment lacking the N-terminal 180 amino acids is particularly preferred. The sequence of this fragment is shown in SEQ ID NO: 3.

The variant of FIH retains 2-OG-dependent oxygenase activity. The 2-OG-dependent oxygenase activity of the variant may be enhanced or reduced compared to FIH having the sequence shown in SEQ ID NO: 1. The variant typically shares at least about 40%, for example at least about 50%, 60%, 70%, 80%, 90% or 95% sequence identity with SEQ ID NO: 1.

The variant of PHD2 retains 2-OG-oxygenase activity. The 2-OG-dependent oxygenase activity of the variant may be enhanced or reduced compared to PHD2 having the sequence shown in SEQ ID NO: 2. The variant of PHD2 typically shares at least about 40%, for example at least about 50%, 60%, 70%, 80%, 90% or 95% sequence identity with SEQ ID NO: 2.

Amino acid substitutions may be made, for example from 1, 2 or 3 to 10, 20 or 30 substitutions. Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Variant polypeptides within the scope of the invention may be generated by any suitable method, for example by gene shuffling (molecular breeding) techniques.

A functional mimetic or derivative of FIH or PHD2 may also be used in an assay of the invention which has hydroxylase activity. Such an active fragment may be included as part of a fusion protein, e.g. including a binding portion for a different, i.e. heterologous, ligand.

The oxygenase and substrate protein for use in an assay of the invention may be chemically modified, e.g. post-translationally modified. For example, they may be glycosylated or comprise modified amino acid residues. They may also be modified by the addition of histidine residues to assist their purification or by the addition of a nuclear localisation sequence to promote translocation to the nucleus or by post translational modification including hydroxylation or phosphorylation.

Substrate

Any suitable substrate may be used in an assay of the invention. Substrates of 2-OG dependent oxygenases are well known in the art. For example, proline is a substrate for proline-3-hydroxylase, collagen is a substrate for lysyl hydroxylase and prolyl-4-hydroxylase, phytanoyl coenzyme A is a substrate of phytanoyl coenzyme A hydroxylase, methylated DNA is a substrate for AlkB and HIF-α polypeptide and ankyrin repeat proteins are substrates of FIH and PHD enzymes.

The substrate may be a naturally occurring protein or a recombinant or synthetic protein. Fragments of substrate proteins which include the site of hydroxylation by the 2-OG oxygenase may be used as substrates in the assay of the invention. For example, the substrate may include the sequence motif DVNA, VN, DVN or IN. The substrate may comprise a sequence motif of the formula:
AA₁-AA₂-N-AA₃
wherein:
AA₁ is D, E, N or Q (preferably D)
AA₂ is V or I (preferably V)
AA₃ is A, V or I (preferably A).
   The asparagine residue (N) is typically hydroxylated by the 2-OG oxygenase.

The assay of the invention may be used to detect novel substrates of a 2-OG oxygenase. In this embodiment a test substrate is used in the assay and the detection of 2-OG oxygenase activity indicates that hydroxylation of the test substance has occurred and that the test substrate is a substrate of the 2-OG oxygenase.

The assay of the invention may also be carried out in the absence of a substrate. In the absence of substrate, 2-OG dependent oxygenases turnover 2-OG at a low rate, for example about 5% of the rate in the presence of substrate. So, the effect of a test agent on the enzyme may be determined even in the absence of substrate.

Derivatisation Reagent

The derivatisation reagent may be any compound that binds to 2-OG to produce a fluorescent product.

The derivatisation reagent typically comprises an aromatic ring with two amine groups on adjacent carbons of the ring as shown in the formula below:

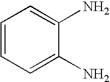

The aromatic ring may be substituted further, for example to make the compound more water soluble or more fluorescent. Fluorescence may be enhanced by increasing the amount of conjugation. This may be achieved, for example, by adding additional aromatic rings such as phenyl or pyridine rings. Accordingly, the derivatisation reagent may be of the formula: 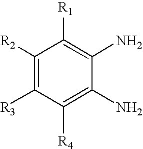
wherein R₁, R₂, R₃ and R4 are the same or different and are each independently H, CO₂H, SO₃H, OCH₃, NO₂, CN, OH, X or CX₃ (wherein X is F, Cl, Br or I), Y or OY (wherein Y is alkyl or alkenyl), CONHNH₂, SONRR′, NRR′ or CON RR′, wherein R and R′ are the same or different and are each independently H, OH, CN, unsubstituted or mono-, di- or poly-substituted aromatic and aromatic heterocylic rings, alkyl or alkoxy, said alkyl or alkoxy being unsubstituted or substituted by one or more of F, Cl, Br and I. R₁ and R4 are preferably H. The alkyl is preferably C₁₋₆ alkyl. The alkoxy is preferably a C₁₋₆ alkoxy. The alkenyl is preferably C₂₋₈ alkenyl Ortho substituents may be joined to form a bicyclic system. R₂ and R₃ may together comprise OCH₂O, or other divalent substituents such as ethylenedioxy. They may, together with the carbon atoms to which they are bonded, form an unsubstituted or mono-, di- or poly-substituted aromatic and aromatic heterocylic ring such as phenyl, diphenyl or pyridine. The aromatic ring is preferably a C₆₋₁₀ aromatic ring and the heterocyclic ring is preferably a 5-10 membered heterocyclic ring. For example, the compound may be of the formula:

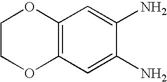

The derivatisation reagent may be, for example, orthophenylenediamine (OPD), 1,2-dimethoxy-4,5-diaminobenzene or 1,2-methylene-dioxy-4,5-diaminobenzene.

Methods for Monitoring Modulation

In order to assay the consumption of 2-OG by a 2-OG dependent oxygenase, such as FIH (and thus measure its catalytic activity), a derivatisation process was developed whereby the 2-OG would form a fluorescent product with ortho-phenylenediamine OPD, or other suitable derivatisation reagent, whereas the succinate, or other products, did not.

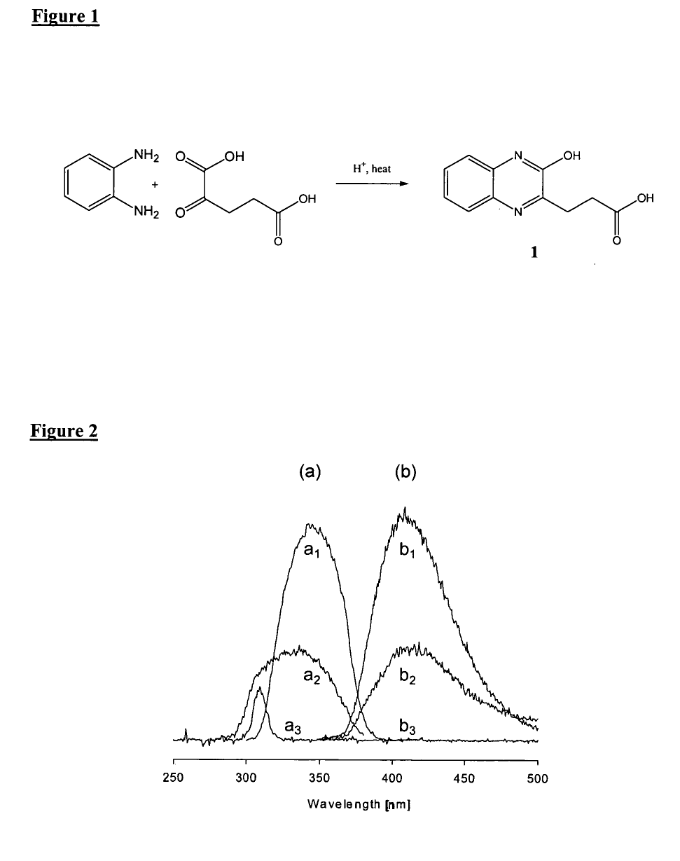

The fluorescent product of the reaction of OPD with the α-ketoacid motif of 2-OG is 3-(2-Carboxyethyl)-2(1H)-quinoxalinone and is illustrated in FIG. 1. This fluorescent product can be readily detected by standard equipment such as that manufactured by for example Molecular Devices, Tecan, BMG Labtechnologies, Jasco and Perkin Elmer and there is extensive precedent demonstrating that the production of fluorescent products can be used in high-throughput screens.

The fluorescent product is generally detected with the excitation filter set as from about 300 nm to about 400 nm, preferably from about 335 to about 345 nm, most preferably at about 340 nm. The emission filter is generally at from about 400 to about 450 nm, preferably from about 415 to about 425 nm, most preferably at about 420 nm.

This assay procedure lends itself to high-throughput formats, such as multi-well plate formats e.g. 96-, 384-, or 1536-well plate formats.

Further, the nature of the fluorescent product can be tuned by modifying the nature of the derivatisation reagent used. For example, the sensitivity of the method may be increased by using either 1,2-dimethoxy-4,5-diaminobenzene, or 1,2-methylenedioxy-4,5 -diaminobenzene.

The precise format of any of the screening or assay methods of the present invention may be varied by those of skill in the art using routine skill and knowledge. The skilled person is well aware of the need to additionally employ appropriate control experiments. Activity is measured by derivatisation of 2-oxoglutarate with ortho-phenylenediamine (OPD) or other aromatic diamines, such as 1,2-dimethoxy-4,5-diaminobenzene or 1,2-methylenedioxy-4,5-diaminobenzene, such that the derivative gives improved sensitivity compared to use of ortho-phenylenediamine (Mühling et al. Journal of Chromatography B (2003) 383-392, Nakamura et al. Chem. Pharm Bull. (1987) 687-692).

Each of the components, where required may be provided either in purified or unpurified form, for example, as cellular extracts or by purification of the relevant component from such extracts. Alternatively, the relevant component can be expressed using recombinant expression techniques and purified for use in the assay.

Alternatively, the components may be expressed recombinantly in a cell for use in cell based assays.

Typically, a polynucleotide encoding the relevant component is provided within an expression vector. Such expression vectors are routinely constructed in the art and may for example involve the use of plasmid DNA and appropriate initiators, promoters, enhancers and other elements, such as for example polyadenylation signals which may be necessary and which are positioned in the correct orientation in order to allow full protein expression. Suitable vectors would be very readily apparent to those of skill in the art. Promoter sequences may be inducible or constitutive promoters depending on the selected assay format. The promoter may be tissue specific.

The assay is carried out under conditions suitable for hydroxylation of the substrate by the 2-OG oxygenase. Accordingly, 2-OG is present in the assay. The assay mixture may also contain iron, preferably ferrous iron. Typically the substrate is mixed with iron and then the enzyme/iron mixture is contacted with the 2-OG/substrate mixture.

Other components may be added to the assay mixture. For example, a reducing agent such as dithiothrietol (DDT), β-mercaptoethanol or N-acetylcysteine may be added to the assay to help maintain enzyme structure and/or catalase may be added to destroy any H₂O₂ that might be produced. However, the assay will work in the absence of a reducing agent or catalase.

The assay is typically carried out at a temperature of from about 25° C. to about 40° C., for example at a temperature of from about 30° C. to about 39° C., or from about 35° C. to about 38° C. or about 37° C. The pH of the assay mixture is typically between about pH 7 to about pH 9, for example from about pH 7.5 to about pH 8. Suitable buffers, such as Tris or HEPES, may be used to maintain the pH of the assay mixture.

Typically, the assay is carried out under normoxic conditions. The assay may also be carried out under conditions in which hydroxylation is reduced or absent, such as under hypoxic conditions, in order to detect modulation of oxygenase activity by an agent which enhances hydroxylation.

Tissue culture cells, organs, animals and other biological systems, which express a 2-OG oxygenase may be used to provide a source of a 2-OG oxygenase. The 2-OG oxygenase may be expressed endogenously. Upregulation of specific endogenous 2-OG oxygenase may be achieved by stimulators of the expression thereof. Such stimulators may be growth factors or chemicals that upregulate specific 2-OG oxygenases. Nucleotide constructs may be introduced into cells to increase production of one or more specific 2-OG oxygenase. Alternatively nucleotide constructs may be introduced into cells so as reduce or abrogate expression of one or more specific 2-OG oxygenase.

Selectivity

In medicinal applications, for example, it is advantageous to modulate 2-OG oxygenase activity selectively, as a single target, or in selected groups. Agents which modulate activity are, therefore, preferably specific i.e. they have an increased or enhanced effect on a first 2-OG oxygenase relative to a second 2-OG oxygenase.

For example, it is recognised that in some circumstances it is advantageous to selectively inhibit FIH or one or more of the aforementioned enzymes, in particular one or more of the HIF prolyl hydroxylase isoforms. For example, N-oxalyl-D-phenylalanine (NOFD) is a selective inhibitor of FIH that has been identified by a method of the invention. Further, in inhibiting some of the above enzymes it may be advantageous not to inhibit FIH and the methods can, for example, be used in a method for discovering PHD2 inhibitors that are not inhibitors of FIH and/or PHD1 and/or PHD3.

The invention provides for the use of such selective inhibitors in the manufacture of a medicament for the treatment of a condition associated with altered, i.e. enhanced or reduced, 2-OG oxygenase activity.

Activities of different enzymes may be compared to detect inhibitors that are selective for a particular 2-OG oxygenase or form of a particular 2-OG oxygenase including but not limited to FIH, AlkB, procollagen prolyl and lysyl hydroxylases, Mina53, the phosphtidylserine receptor, 2-OG oxygenases that have been characterized as JmjC proteins, according to the SMART database, and/or any of the PHD enzymes including PHD1, PHD2 and PHD3.

It is also possible, using the method of the invention to identify selective inhibitors when the substrate of one or more of the enzymes being tested is unknown. In this embodiment, generally it will be one or more of the enzymes that it is wished not to inhibit that has an unknown substrate. The effect of a test agent on activity of an oxygenase may be determined in the absence of a substrate by determining whether or not the test agent affects, for example inhibits or stimulates, the rate of turnover of 2-OG by the oxygenase.

Test Compounds

Compounds which may be screened using the assay methods described herein may be natural or synthetic chemical compounds used in drug screening programmes. Extracts of plants, microbes or other organisms which contain several characterised or uncharacterised components may also be used.

Combinatorial library technology (including solid phase synthesis and parallel synthesis methodologies) provides an efficient way of testing a potentially vast number of different substances for ability to modulate an interaction. Such libraries and their use are known in the art, for all manner of natural products, small molecules and peptides, among others. The use of peptide libraries may be preferred in certain circumstances.

Potential inhibitor compounds (i.e. antagonists) may be polypeptides, small molecules such as molecules from commercially available combinatorial libraries, or the like. Small molecule compounds which may be used include 2-OG analogues, or substrate analogues, which inhibit the action of the enzyme.

Potential promoting agents may be screened from a wide variety of sources, particularly from libraries of small compounds which are commercially available. Oxygen-containing compounds may be included in candidate compounds to be screened, for example 2-OG analogues.

A test compound which increases, potentiates, stimulates, disrupts, reduces, interferes with or wholly or partially abolishes hydroxylation of the substrate and which may thereby modulate activity, may be identified and/or obtained using the assay methods described herein.

Agents which increase or potentiate hydroxylation (i.e. agonists), may be identified and/or obtained under conditions which, in the absence of a positively-testing agent, limit or prevent hydroxylation. Such agents may be used to potentiate, increase, enhance or stimulate the activity of a 2-OG oxygenase.

In various aspects, the present invention provides an agent or compound identified by a screening method of the invention to be a modulator of 2-OG oxygenase activity e.g. a substance which inhibits or reduces, increases or potentiates the activity of a 2-OG oxygenase.

Following identification of a modulator, the substance may be purified and/or investigated further (e.g. modified) and/or manufactured. A modulator may be used to obtain peptidyl or non-peptidyl mimetics, e.g. by methods well known to those skilled in the art and discussed herein. A modulator may be modified, for example to increase selectively, as described herein. It may be used in a therapeutic context as discussed below.

For therapeutic treatment, the compound may be used in combination with any other active substance, e.g. for anti-tumour therapy another anti-tumour compound or therapy, such as radiotherapy or chemotherapy.

Suitable test compounds include D stereoisomers of formula (I): 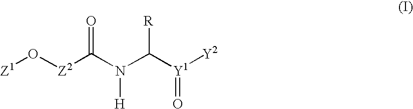
wherein
Y² is selected from —OR′ and —NR′R″ wherein R′ is hydrogen, or unsubstituted C₁₋₄ alkyl and R″ is hydrogen, hydroxy or unsubstituted C₁₋₄ alkyl;
Y¹ is selected from —C—, —S— and —S(O)—;
Z² is selected from —C(O)— and —NR′— wherein R″ is selected from hydrogen, hydroxy or unsubstituted C₁₋₄ alkyl;
Z¹ is selected from hydrogen and unsubstituted C₁₋₄ alkyl; and
R is a side chain of a naturally occurring amino acid.

Preferably Y¹ is —C—and Y² is —OH or —NH₂. Most preferably Y¹ is —C— and Y² is —OH.

Preferably Z2 is —C(O)— or —NR″— wherein R″ is hydrogen, methyl or ethyl. More preferably Z² is —C(O)— or —NH—. Preferably Z¹ is hydrogen, methyl or ethyl, more preferably hydrogen. Most preferably Z² is —C(O)— and Z¹ is hydrogen, methyl or ethyl.

Preferably R is a side chain of alanine, valine, leucine or phenylalanine. Preferably R is a side chain of valine, leucine or phenylalanine. More preferably R is a side chain of phenylalanine, i.e. —CH₂Ph.

Particularly suitable compounds include:
N-oxalyl-D-tyrosine; and
N-oxalyl-D-phenylalanine.

The compounds which are acids can be present in the form of salts, such as sodium salts. The compounds may also be present in the form of derivatives such as the dimethyl ester, diethyl ester, monoethyl ester or di- or mono-amide. In certain instances these derivatives may be preferred, for example when inhibition of the enzyme within a cell of an organism is required.

An exemplary synthetic scheme used to test compounds is shown below in Scheme 1. Here an amino acid is reacted with an oxalyl chloride in order to produce a compound of the invention. In this scheme the amino acid used is phenylalanine, although it will be apparent that the same general reaction will occur with other amino acids. The first reaction yields a protected compound of the invention (the dimethyl ester form). The diacid form is easily generated through reaction with aqueous sodium hydroxide.

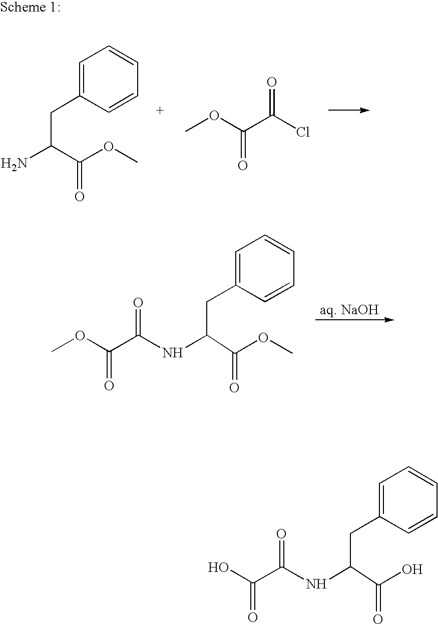

Compounds in which X is —O— or —S— or Z is other than —CO—CO—OH may by synthesised as described in Mole et al. (2003) Bioorg. Med. Chem. Lett. 13, 2677-2680 and Cunliffe et al. J. Med. Chem. (1992) 35 2652-2658.

Therapeutic Applications

A compound, substance or agent which is found to have the ability to affect the hydroxylase activity of a 2-OG dependent oxygenase has therapeutic and other potential in a number of contexts, as discussed. For therapeutic treatment, such a compound may be used in combination with any other active substance, e.g. for anti-tumour therapy with another anti-tumour compound or therapy, such as radiotherapy or chemotherapy.

An agent identified using one or more primary screens (e.g. in a cell-free system) as having ability to modulate the activity of a 2-OG dependent oxygenase on a substrate may be assessed further using one or more secondary screens.

Generally, an agent, compound or substance which is a modulator according to the present invention is provided in an isolated and/or purified form, i.e. substantially pure. This may include being in a composition where it represents at least about 90% active ingredient, more preferably at least about 95%, more preferably at least about 98%. Any such composition may, however, include inert carrier materials or other pharmaceutically and physiologically acceptable excipients, such as those required for correct delivery, release and/or stabilisation of the active agent. As noted below, a composition according to the present invention may include in addition to an modulator compound as disclosed, one or more other molecules of therapeutic use, such as an anti-tumour agent.

Products Obtained by Assays of the Invention

The invention further provides compounds obtained by assay methods of the present invention, and compositions comprising said compounds, such as pharmaceutical compositions wherein the compound is in a mixture with a pharmaceutically acceptable carrier or diluent. The carrier may be liquid, e.g. saline, ethanol, glycerol and mixtures thereof, or solid, e.g. in the form of a tablet, or in a semi-solid form such as a gel formulated as a depot formulation or in a transdermally administrable vehicle, such as a transdermal patch.

The 2-OG oxygenases are involved in various pathways that are of medicinal and/or commercial importance. Their roles include:

The hydroxylation of prolyl and lysyl residues in collagen biosynthesis as catalysed by prolyl-4-hydroxylase and lysyl hydroxylase. Modulators of prolyl-4-hydroxylase or lysyl hydroxylase according to the invention may be used in methods of treating connective tissue diseases such as systemic lupus erythematosus (SLE), scleroderma, vasculitis and myositis.

The oxidation of fatty acid metabolites including phytanoyl coenzyme A is catalysed by phytanoyl Coenzyme A hydroxylase. Modulators of phytanoyl coenzyme A may be used in methods of treating Refsum's disease.

The oxidative demethylation of methylated DNA is catalysed by Alk-B. Modulators of Alk B activity may be used in methods of treating or preventing diseases arising from DNA damage, such as damage caused by smoking and/or atmospheric pollution.

The biosynthesis of hydroxylated amino acids, including 4-hydroxyproline and 3-hydroxyproline as catalysed by proline-3-hydroxylase. A recombinant organism using a 2-OG oxygenase is used commercially for the production of 4-hydroxyproline. Agents which activate or potentiate 2-OG oxygenase activity are, therefore useful in the production of 4-hydroxyproline.

Various steps in the biosynthesis of commercially and medicinally important antibiotics are catalysed by 2-OG oxygenases. Enzymes so involved include clavaminic acid synthase, deacetoxycephalosporin C synthase, and carbapenem synthase. Agents which activate or potentiate activity of such 2-OG oxygenases are, therefore, useful in the production of antibiotics.

2-OG oxygenases catalyse many other reactions and are involved in the medicinally important hypoxic response process.

Reduced dioxygen concentration in the tissues of multicellular organism triggers the hypoxic response which attempts to restore normoxia by improving the supply of oxygen. The response involves an array of genes including erythropoietin and vascular endothelial growth factor and is mediated by a specific α,β-heterodimeric transcription factor, hypoxia-inducible factor (HIF), the α-subunit of which is upregulated under hypoxic conditions. Since the genes involved in the hypoxic response include those involved in angiogenesis, modulation of the hypoxic response is of interest from the perspectives of developing new therapies for both cancer and cardiovascular disease.

Both the levels and activity of HIF-α are regulated by 2-OG oxygenases. Under normoxic conditions, two different but related dioxygenases, prolyl hydroxylase domain (PHD also known as EGLN and HPH) enzymes and factor inhibiting hypoxia-inducible factor (FIH) inhibit hypoxic responses by catalysing the post-translational hydroxylation of HIF-α.

Both HIF-1α and HIF-2α contain a central oxygen dependent degradation domain (ODDD). Pro-402 and Pro-564 (in HIF-1α) are situated in two sub-domains of the ODDD. Hydroxylation of these residues, by PHD isoforms, enables binding of HIF-α to the pVHL-Elongin B/C (VBC) complex which recruits an E3 ubiquitin ligase, mediating ubiquitination of HIF-α and its consequent proteasomal destruction. The prolyl residues forms part of a conserved LXXLAP motif and modification of either residue can independently promote degradation.

Upregulation of HIF is involved in the development of tumours associated with defects in the von Hippel/Lindau tumour suppressor protein, pVHL. The binding of hydroxylated HIF-α to p VHL relies upon two hydrogen bonds involving the alcohol of the trans-4-hydroxylated prolyl residue in the former to the side-chains of a serinyl and histidinyl residues in pVHL.

Under hypoxic conditions, cytoplasmic HIF-α translocates into the nucleus and binds to HIF-β, forming the active heterodimer which then acts in conjunction with nuclear coactivators, including p300. Hydroxylation by FIH at the β-carbon of a conserved asparagine residue (Asn-803 in HIF-1α) in the C-terminal activation domain (CAD) of HIF prevents binding of HIF to the CH1 domain of p300, a nuclear co-activator protein involved in transcription. Modulators of 2-OG oxygenases are thus useful in the treatment or prevention of cancer.

The invention further provides a method of treatment which includes administering to a patient an agent which interferes with 2-OG oxygenase activity. Such agents may include inhibitors of 2-OG oxygenase activity.

The therapeutic/prophylactic purpose may be related to the treatment of a condition associated with reduced or suboptimal or increased 2-OG oxygenase levels or activity, or conditions in which have normal 2-OG oxygenase levels, but where an modulation in activity such as an increase or decrease in 2-OG oxygenase activity is desirable such as:
(i) ischaemic conditions, for example organ ischaemia, including coronary, cerebrovascular and peripheral vascular insufficiency. The therapy may be applied in two ways; following declared tissue damage, e.g. myocardial infarction (in order to limit tissue damage), or prophylactically to prevent ischaemia, e.g. promotion of coronary collaterals in the treatment of angina;
(ii) cancer; HIF-α is commonly up-regulated in tumour cells and has major effects on tumour growth and angiogenesis;
(iii) inflammatory disorders; and
(iv) immune disorders such as diabetes.

Modulating 2-OG oxygenase activity in a person, an organ, or a group of cells may be exploited in different ways to obtain a therapeutic benefit.

An agent of the invention may promote cell survival or proliferation and/or inhibit apoptosis (such as might be achieved by reducing interaction of p53 and ASPP1 or 2 or increasing interaction of p53 with iASPP or by reducing interaction of the tumour suppressor proteins p16 or p18 with cyclin dependent kinases). Such an agent is useful in the treatment of ischaemia, hypoxia or otherwise damaged tissues.

An agent of the invention may inhibit survival of tumour cells (such as might be achieved by promoting interaction of p53 and ASPP1 or 2 or decreasing interaction of p53 with iASPP or by promoting interaction of the tumour suppressor proteins p16 or p18 with cyclin dependent kinases). Such an agent is active against cancerous tissues.

An agent of the invention may regulate inflammation and immunity (such as might be achieved by reducing or increasing the interaction between NFκB proteins such as p105 and IκB-α and the p50/p65 transcriptional complex).

A therapeutically effective amount of an agent is typically administered to a subject in need thereof. A therapeutically effective is an amount which ameliorates the symptoms of the condition or lessens the suffering caused to the subject by the condition.

Pharmaceutical Compositions

In various further aspects, the present invention thus provides a pharmaceutical composition, medicament, drug or other composition for such a purpose, the composition comprising one or more agents, compounds or substances as described herein, including inhibitors of 2-OG oxygenase activity, the use of such a composition in a method of medical treatment, a method comprising administration of such a composition to a patient, e.g. for treatment (which may include preventative treatment) of a medical condition as described above, use of such an agent compound or substance in the manufacture of a composition, medicament or drug for administration for any such purpose, e.g. for treatment of a condition as described herein, and a method of making a pharmaceutical composition comprising admixing such an agent, compound or substance with a pharmaceutically acceptable excipient, vehicle or carrier, and optionally other ingredients.

In one embodiment the method for providing a pharmaceutical composition may typically comprise:
(a) identifying an agent by an assay method of the invention; and
(b) formulating the agent thus identified with a pharmaceutically acceptable excipient.

The pharmaceutical compositions of the invention may comprise an agent, polypeptide, polynucleotide, vector or antibody according to the invention and a pharmaceutically acceptable excipient.

The agent may be used as sole active agent or in combination with one another or with any other active substance, e.g. for anti-tumour therapy another anti-tumour compound or therapy, such as radiotherapy or chemotherapy.

Whatever the agent used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

An agent or composition may be administered alone or in combination with other treatments, either simultaneously or sequentially dependent upon the condition to be treated, e.g. as described above.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. In particular they may include a pharmaceutically acceptable excipient. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Liposomes, particularly cationic liposomes, may be used in carrier formulations. Examples of techniques and protocols mentioned above can be found in Remington's Pharmaceutical Sciences, 16th edition, Osol, A. (ed), 1980.

The substance or composition may be administered in a localised manner to a particular site or may be delivered in a manner in which it targets particular cells or tissues, for example using intra-arterial stent based delivery.

Targeting therapies may be used to deliver the active substance more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

In a further embodiment the invention provides for the use of an agent of the invention in the manufacture of a medicament for the treatment of a condition associated with increased or decreased 2-OG oxygenase levels or activity. The condition may, for example, be selected from the group consisting of ischaemia, cancer, and inflammatory and immune disorders.

All the documents cited herein are incorporated herein by reference.

The following Examples illustrate the invention.

### EXAMPLES

### Example 1

### Materials

OPD was bought from Acros Organics and recrystallised from heptane and petroleum ether (120-140). DTT was from Melford Laboratories. Catalase and iron ammonium sulphate were from Sigma. FIH and GST-tagged HIF-1α 786-826 were prepared as described previously (Hewitson, et al. J. Biol. Chem. (2002) 277: 26351-26355).

Scanning emission and excitation spectra were recorded on a Perkin Elmer LK-50B spectrometer.

### Example 2

### Detection of FIH Activity

The assay of FIH activity was carried out by mixing 1 mM DTT, 0.6 mg/ml catalase, 2-OG, substrate and 50 mM Tris/HCl pH 7.5 to a final volume of 88 μl warming to 37° C. for 5 minutes in a water bath. Simultaneously, the enzyme and iron (prepared as 500 mM stock in 20 mM HCl, and diluted with water) were mixed at room temperature for 3 minutes. Reaction was initiated by addition of 12 μl of enzyme/iron mix to the substrate/cofactor mix. The reaction was stopped by addition of 200 μl 0.5M HCl; derivatisation was then achieved by the addition of 100 μl 10 mg/ml OPD in 0.5M HCl, and heating for 10 minutes at 95° C. in a heating block. After centrifugation at top speed in a bench microfuge for 5 minutes, the supernatant (50 μl) was made basic by the addition of 30 μl 1.25M NaOH and the fluorescence was measured on a Novostar (BMG Labtechnologies Ltd.) with the excitation filter at 340 nm and the emission filter at 420 nm.

The product of the reaction between OPD and 2-oxoglutarate was characterised by ¹H and ¹³C nmr and by mass spectrometry, confirming that the cyclisation reaction proceeded as expected to give the fluorescent product.

NMR Data:

¹H-NMR (DMSO-D₆) δ [ppm]:7.71 (m, 1H, CH_{Ar}), 7.50 (m, 1H, CH_{Ar}), 7.30 (m, 2H, CH_{Ar}), 3.03 (t, 2H, CH₂), 2.74 (t, 2H, CH₂)

¹³C-NMR (DMSO-D₆) δ [ppm]:174.7 (C_{quart}), 161.1 (C_{quart}), 155.4 (C_{quart}), 132.5 (C_{quart/Ar}), 132.3 (C_{quart/Ar}), 130.3 (CH_{Ar}), 128.9 (CH_{Ar}), 123.9 (CH_{Ar}), 116.1 (CH_{Ar}), 30.4 (CH₂), 28.5 (CH₂).

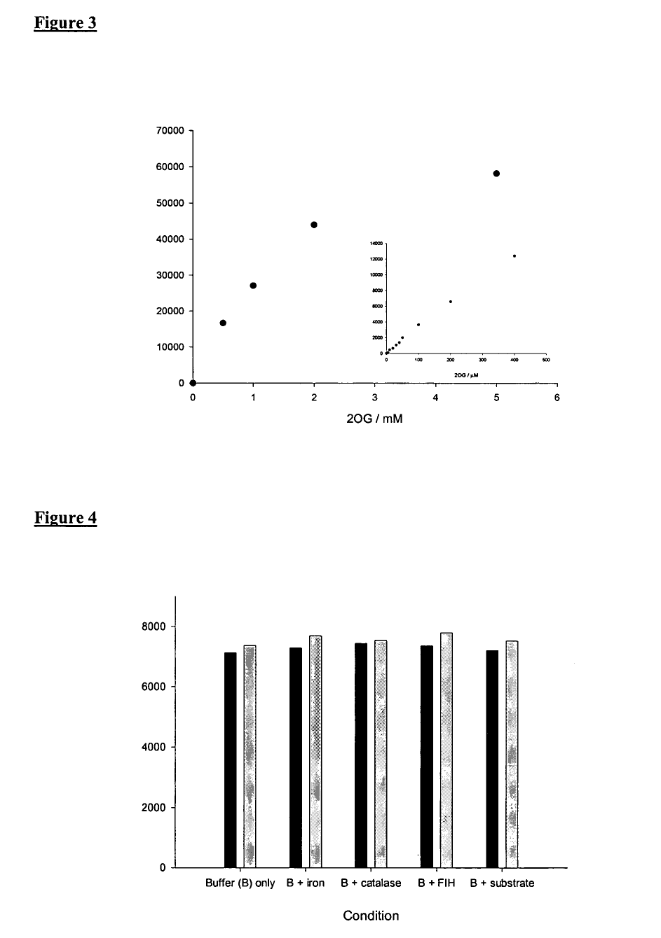

Fluorescence spectra of the product (1 in FIG. 1) revealed that the maximum response was obtained under basic conditions, exciting at 340 nm and measuring the emission at 420 nm (FIG. 2).

Linearity of the developed fluorescence with respect to the concentration of 2-OG was demonstrated up to 1 mM 2-OG (FIG. 3), and it was shown that the presence of GST HIF-1α 786-826, FIH, catalase, DTT, and Fe, both separately and in combination had no appreciable effect on the development of fluorescence up to the highest concentrations used in the assay procedure reported here (FIG. 4).

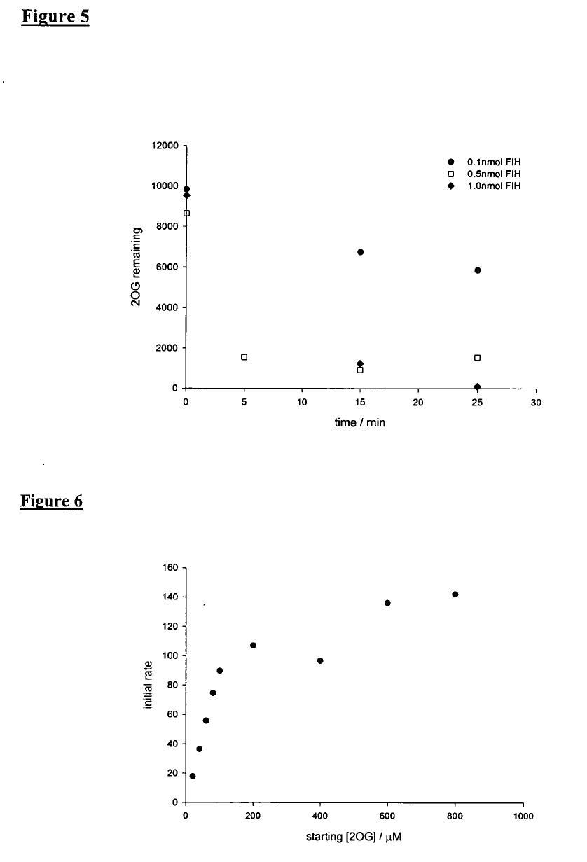

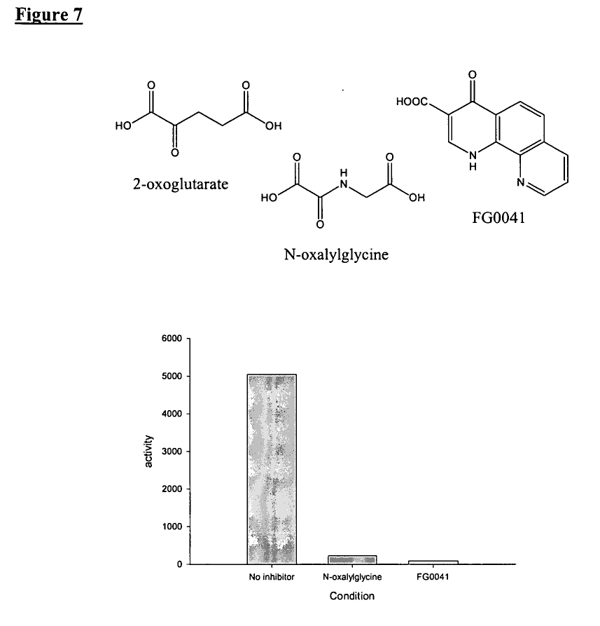

The initial rate of the reaction was seen to vary with enzyme (FIG. 5) and 2-OG concentration (FIG. 6).

### Example 3

### Inhibition of FIH Activity

It was also shown (FIG. 7) that known inhibitors of 2-OG oxygenases N-oxalylglycine (Epstein, et al. Cell (2001) 107:43-54), and FG0041(Ivan, et al. Proc. Natl. Acad. Sci. U. S. A. (2002) 99: 13459-13464) eliminated the 2-OG consumption by FIH in the presence of prime substrate and 2-OG. FG0041 has been previously reported as a PHD inhibitor, but not as an FIH inhibitor. The observation that it inhibits both FIH and the PHD enzymes has consequences for its development, and that of structurally related compounds such as derivatives of FG0041, as pharmaceuticals modulating the hypoxic response.

### Example 4

### Assay Conditions for PHD2

The PHD2 used was a form of the enzyme that has had the first 180 amino acids (including those containing a MYND finger motif) removed. Thus the enzyme, after removal of the affinity tag, has a N-terminus of GSHPNGQTKPLP; we have found this form of PHD2 to be preferred in the in vitro assays. The enzyme was expressed from the pET28a(+) vector (Novagen), with the gene cloned between NheI and BamHI restriction sites. Growth and induction were using standard techniques, in that the pET28a(+) vector was transformed into BL21(DE3) Escherichia coli cells, and grown in 2TY medium at 37° C. until the absorbance of the solution of 600 nm was 0.8. IPTG was added to a final concentration of 0.5 mM and growth was continued for three hours. Affinity purification using Novagen His-Bind resin was as per the manufacturers instructions.

The assay was carried out as for the FIH, but the buffer used was 50 mM HEPES pH7.0.

### Example 5

### Synthesis of Inhibitors

The following procedure (Cunliffe et al. J. Med. Chem. (1992) 35 2652-2658) was used to make the diester of NOFD (N-oxalyl-D-phenylalanine).

To a stirred solution of 10 mmol of phenylalanine methyl ester in 10 ml of toluene 10 mmol (1.23 g) methyl oxalylchloride was added and heated to reflux until no further HCl gas evolved (usually 4-6h). The solvent was then evaporated in vacuo and the product purified by column chromatography.

δ_{H} (300 MHz, CDCl₃):3.1 [t, CHCH₂Ar], 3.7-3.8 [2xs, OCH₃], 4.85 [q, NHCHCO₂Me], 7-7.25 [br m, CH₂C₆H₅], 7.45 [br s, NH]. δ_{C} (75 MHz, CDCl₃): 38.0 [t, CH₂Ar], 53.0 [q, CH₂CH(CO₂Me)NH], 54.0-54.1 [2xs, OCH₃], 127.8-135.5 [4xs, C₆H₅], 156.1-171.1 [3xs, CO].

In order to obtain the diacid N-oxalyl derivative of the dimethylester derivative of N-oxalylphenylalanine, the following procedure was used. The ester compound was treated for 60 minutes with a sufficient amount of 2 N aqueous NaOH solution ensuring 1.1 equivalents of sodium hydroxide for the sum of the ester functions to be cleaved in the compound. The reaction was percolated through a column of “Amberlite IR 120 H” ion exchange resin (previously washed with water to about pH 4) and eluted with water until the pH raised to 4 again. The water was evaporated in vacuo and the residue dried under vacuum.

δ_{H} (300 MHz, D₂O):3.0-3.2 [n, CHCH₂Ar], 4.63 [q, NHCHCO₂Me], 7-7.25 [br m, CH₂C₆H₅]. δ_{C} (75 MHz, D₂O):36.5 [t, CH₂Ar], 54.6 [q, CH₂CH(CO₂Me)NH], 127.5-136.5 [4xs, C₆H_{5], 160.0}-174.2 [3xs, CO].

### Example 6

### Selective Inhibition of 2-OGs

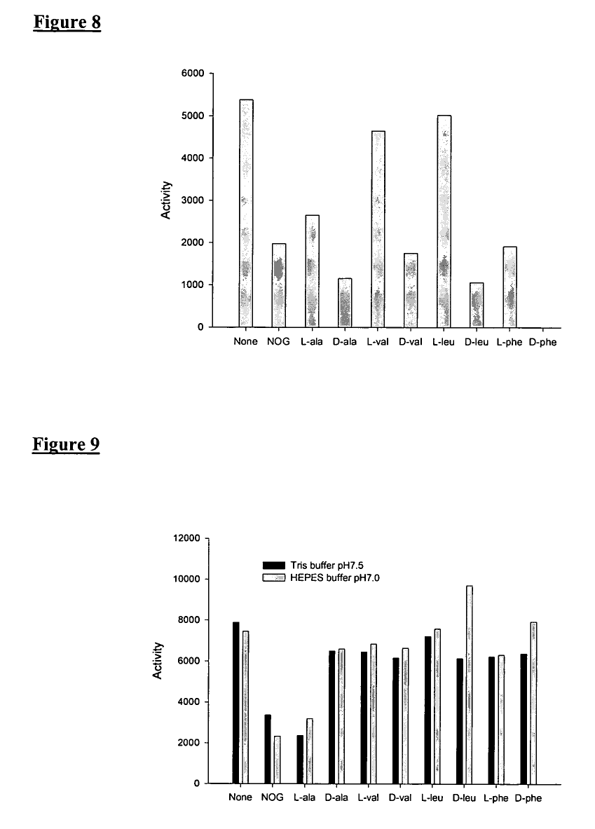

The effect of N-oxalyl amino acid inhibitors (1 mM) on the activity of FIH is shown in FIG. 8. It can be seen that N-oxalyl-(D)-phenylalanine (NOFD) derivative is the most potent inhibitor of FIH in the series, with an IC₅₀ of 7 μM, significantly more potent than N-oxalylglycine which has an IC₅₀ of 40 μM.

The effect of N-oxalyl amino acid inhibitors (1 mM) on the activity of PHD2 in both Tris and HEPES buffers is shown in FIG. 9. It can be seen that NOFD, which is a potent inhibitor of FIH, causes little, if any, inhibition of PHD2.

1. A method for detecting 2-oxoglutarate oxygenase activity, which method comprises the steps of:
(i) contacting a 2-oxoglutarate oxygenase and a substrate of the 2-oxoglutarate oxygenase in the presence of 2-oxoglutarate;
(ii) adding a derivatisation reagent capable of forming a fluorescent product with 2-oxoglutarate; and
(iii) detecting the fluorescent product produced by the reaction between the derivatisation reagent and 2-oxoglutarate, if any, thereby detecting 2-oxoglutarate oxgenase activity.

2. A method according to claim 1, wherein the derivatisation agent is an aromatic diamine, or a derivative of an aromatic diamine.

3. A method according to claim 2, wherein the derivatisation agent is ortho-phenylenediamine, 1,2-dimethoxy-4,5-diaminobenzene or 1,2-methylenedioxy-4,5-diaminobenzene.

4. A method according to claim 1, wherein the 2-oxoglutarate oxygenase is factor inhibiting hypoxia inducible factor (FIH) or a prolyl hydroxylase domain containing (PHD) enzyme.

5. A method according to claim 4, wherein the PHD enzyme is PHD1, PHD2 or PHD3.

6. A method according to claim 5, wherein the PHD enzyme is a fragment of PHD2 having 2-oxoglutarate activity.

7. A method according to claim 6 wherein the fragment of PHD2 is a truncated form of PHD2 which lacks the first 180 N-terminal amino acids.

8. A method according to claim 1, wherein the 2-oxoglutarate oxygenase is selected from the group consisting of AlkB, phytanoyl CoA hydroxylase, trimethyllysine hydroxylase, γ-butyrobetaine hydroxylase, phosphatidylserine receptor, Mina-53, pro-collagen prolyl hydroxylase and pro-collagen lysyl hydroxylase.

9. A method for determining whether a test agent modulates activity of a 2-oxoglutarate dependent oxygenase comprising the steps of:
(i) contacting a 2-oxoglutarate oxygenase, a test agent and optionally a substrate of the 2-oxyglutarate oxygenase in the presence of 2-oxoglutarate;
(ii) adding a derivatisation reagent capable of forming a fluorescent product with 2-oxoglutarate;
(iii) detecting the fluorescent product produced by the reaction between the derivatisation reagent and 2-oxoglutarate, if any; and
(iv) comparing the amount of fluorescent product detected to the amount of fluorescent product detected in the absence of the test agent,
thereby determining whether the test agent modulates activity of the 2-oxygenase.

10. A method according to claim 9, wherein said test agent inhibits 2-oxoglutarate oxygenase activity.

11. A method according to claim 9, wherein said test agent activates 2-oxoglutarate oxygenase activity.

12. A method according to claim 9, wherein the substrate of said 2-oxoglutarate oxygenase is unknown.

13. A method according to claim 9, wherein the derivatisation agent is an aromatic diamine, or a derivative of an aromatic diamine.

14. A method according to claim 9, wherein the derivatisation agent is ortho-phenylenediamine, 1,2-dimethoxy-4,5-diaminobenzene or 1,2-methylenedioxy-4,5-diaminobenzene.

15. A method according to claim 9, wherein the 2-oxoglutarate oxygenase is factor inhibiting hypoxia inducible factor (FIH) or a prolyl hydroxylase domain (PHD) containing enzyme.

16. A method according to claim 15, wherein the PHD enzyme is PHD1, PHD2 or PHD3.

17. A method according to claim 16, wherein the PHD enzyme is a fragment of PHD2 having 2-oxoglutarate activity.

18. A method according to claim 17, wherein the fragment of PHD2 is a truncated form of PHD2 which lacks the first 180 N-terminal amino acids.

19. A method for determining whether a test agent is a selective modulator of a first 2-oxyglutarate oxygenase, comprising the steps of:
(i) contacting a first 2-oxoglutarate oxygenase, a test agent and optionally a substrate of the first 2-oxoglutarate oxygenase in the presence of 2-oxoglutarate;
(ii) adding a derivatisation reagent capable of forming a fluorescent product with 2-oxoglutarate;
(iii) detecting the fluorescent product produced by the reaction between the derivatisation reagent and 2-oxoglutarate, if any;
(iv) comparing the amount of fluorescent product detected to the amount of fluorescent product detected in the absence of the test agent;
(v) contacting a second 2-oxoglutarate oxygenase, a test agent and optionally a substrate of the second 2-oxoglutarate oxygenase in the presence of 2-oxoglutarate;
(vi) adding a derivatisation reagent capable of forming a fluorescent product with 2-oxoglutarate;
(vii) detecting the fluorescent product produced by the reaction between the derivatisation reagent and 2-oxoglutarate, if any; and
(viii) comparing the amount of fluorescent product detected to the amount of fluorescent product detected in the absence of the test agent;
thereby determining whether the test agent is a selective modulator of the first 2-oxoglutarate oxygenase, wherein the test agent is a selective modulator of the first 2-oxoglutarate oxygenase if it modulates activity of the first 2-oxoglutarate oxygenase but does not substantially modulate activity of the second 2-oxoglutarate oxygenase.

20. A method according to claim 19, wherein said test agent inhibits 2-oxoglutarate oxygenase activity.

21. A method according to claim 19, wherein said test agent activates 2-oxoglutarate oxygenase activity.

22. A method according to claim 19, wherein the substrate of said first and/or said second 2-oxoglutarate oxygenase is unknown.

23. A method according to claim 19, wherein the derivatisation agent is an aromatic diamine, or a derivative of an aromatic diamine.

24. A method according to claim 19, wherein the derivatisation agent is ortho-phenylenediamine, 1,2-dimethoxy-4,5-diaminobenzene or 1,2-methylenedioxy-4,5-diaminobenzene.

25. A method according to claim 19, wherein the first 2-oxoglutarate oxygenase is factor inhibiting hypoxia inducible factor (FIH) or a prolyl hydroxylase domain (PHD) containing enzyme.

26. A method according to claim 25, wherein the PHD enzyme is PHD1, PHD2 or PHD3.

27. A method according to claim 26, wherein the PHD enzyme is a fragment of PHD2 having 2-oxoglutarate activity.

28. A method according to claim 27, wherein the fragment of PHD2 is a truncated form of PHD2 which lacks the first 180 N-terminal amino acids.

29. An agent which modulates 2-oxoglutarate activity identified by a method for detecting 2-oxoglutarate oxygenase activity or determining whether a test agent modulates activity of a 2-oxoglutarate depenent oxygenase.

30. A method of treating a condition associated with increased or reduced 2-oxoglutatrate activity or a condition in which it is desired to modulate 2-oxoglutarate activity, comprising administering a therapeutically effective amount of an agent according to claim 29 to an individual having the condition.

31. A method according to claim 30 wherein the condition is selected from the group consisting of ischemia, wounding, auto-, allo- and xeno-transplantation, systemic high blood pressure, cancer, inflammatory disorders and diabetes.


SEQUENCE LISTING (3 sequences)

SEQ ID NO: 1 - protein, 349 aa, Homo sapiens
MAATAAEAVASGSGEPREEAGALGPAWDESQLRSYSFPTRPIPRLSQSDPRAEELIENEEPVVLTDTNLVYPALKWDLEYLQENIGNGDFSVYSASTHKFLYYDEKKMANFQNFKPRSNREEMKFHEFVEKLQDIQQRGGEERLYLQQTLNDTVGRKIVMDFLGFNWNWINKQQGKRGWGQLTSNLLLIGMEGNVTPAHYDEQQNFFAQIKGYKRCILFPPDQFECLYPYPVHHPCDRQSQVDFDNPDYERFPNFQNVVGYETVVGPGDVLYIPMYWWHHIESLLNGGITITVNFWYKGAPTPKRIEYPLKAHQKVAIMRNIEKMLGEALGNPQEVGPLLNTMIKGRYN

SEQ ID NO: 2 - protein, 426 aa, Homo sapiens
MANDSGGPGGPSPSERDRQYCELCGKMENLLRCSRCRSSFYCCKEHQRQDWKKHKLVCQGSEGALGHGVGPHQHSGPAPPAAVPPPRAGAREPRKAAARRDNASGDAAKGKVKAKPPADPAAAASPCRAAAGGQGSAVAAEAEPGKEEPPARSSLFQEKANLYPPSNTPGDALSPGGGLRPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF

SEQ ID NO: 3 - protein, 249 aa, Homo sapiens
GSHPNGQTKPLPALKLALEYIVPCMNKHGICVVDDFLGKETGQQIGDEVRALHDTGKFTDGQLVSQKSDSSKDIRGDKITWIEGKEPGCETIGLLMSSMDDLIRHCNGKLGSYKINGRTKAMVACYPGNGTGYVRHVDNPNGDGRCVTCIYYLNKDWDAKVSGGILRIFPEGKAQFADIEPKFDRLLFFWSDRRNPHEVQPAYATRYAITVWYFDADERARAKVKYLTGEKGVRVELNKPSDSVGKDVF